(19) United States
(12) Patent Application Publication
(10) Pub. No.: US 2006/0051440 A1
(43) Pub. Date: Mar. 9, 2006

(54) Factors that bind intestinal toxins

(76) Inventors: Joel Moss, Bethesda, MD (US); Masatoshi Noda, Yotsukaido (JP)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/526,820

(22) PCT Filed: Jan. 31, 2003
(86) PCT No.: PCT/US03/02882

§ 371 Date: Mar. 3, 2005

Related U.S. Application Data
(60) Provisional application No. 60/409,742, filed on Sep. 10, 2002.

(51) Int. Cl. A61K 36/185; A61K 31/7048; A61K 31/496; A61K 31/353; A61K 31/65

(52) U.S. Cl. 424/778; 514/27; 514/154; 514/253.08; 514/456

(57) ABSTRACT

Methods for neutralizing bacterial toxins such as Shiga toxins and cholera toxins are disclosed. In a particular embodiment, a method is provided for treating a subject suffering from an infection caused by an Stx-producing organism by administering a therapeutically effective amount of a hop bract tannin obtained from Humulus lupulus. Also provided are methods for isolating polyphenolic compounds that bind Stx molecules, and methods for detecting the presence of Stx molecules in a biological sample. In a disclosed embodiment, a subject infected with a Shiga toxin-producing E. coli strain is treated by enterically administering a high molecular weight fraction of hop bract extract to the subject.

### PRIORITY CLAIM

This claims the benefit of U.S. Provisional Application No. 60/409,742, filed Sep. 10, 2002, which is incorporated by reference in its entirety.

### FIELD

The invention relates to diagnosis and treatment of bacterial infections and their symptoms. More specifically, the invention concerns the use of compositions derived from hop bracts to neutralize bacterial toxins, such as Shiga-toxins.

### BACKGROUND

In a large number of enteric diseases, caused by bacterial infection, toxins elaborated by the organism appear to be responsible for the clinical presentation. Thus, vaccination against the toxic products of the organism may be sufficient for prevention of disease. For example, for tetanus, diphtheria and pertussis, immunization prevents the overt signs of infection. However, for enteric diseases, such as cholera and certain E. coli infections, immunization is not as effective because symptoms largely result from the effects of toxins on intestinal cells.

Strong epidemiological evidence supports an association of Shiga toxin-1 (Stx1)-producing Escherichia coli strains (STEC) with outbreaks of hemorrhagic colitis, hemolytic uremic syndrome (HUS), and encephalopathy. Stx1 is the dominant virulence factor in diseases caused by STEC. In general, antibiotics are used for STEC infections. However, following antibiotic administration, STEC, such as E. coli O157:H7, often produce massive amounts of Stx1, leading to a worsening of the clinical condition. Furthermore, although antibiotics have saved the lives of many patients, their administration has resulted in new drug-resistant bacteria such as methicillin-resistant Staphylococcus aureus (RSA) and vancomycin-resistant enterobacteria (VRE), leaving some conditions untreatable.

The biological activities of Stx1 are well characterized. It is cytotoxic for Vero cells and a certain line of HeLa cells, lethal for mice and other small rodents, and enterotoxic, causing fluid accumulation in rabbit ileal loop assays. Stx1 consists of two subunits, an A-subunit and five B-subunits. The A-subunit (StxA) is a 33-kDa enzyme that blocks protein synthesis in eukaryotic cells through its RNA N-glycosidase activity. StxA cleaves an N-glycosidic bond of adenosine at position 4,324 from the 5′-terminus of the 28S ribosomal RNA [60S ribosomal subunit in rabbit reticulocytes]. The Stx1 B-subunits (StxB) bind to Gb3 globotriaosylceramide on the cell surface, facilitating STXA translocation into the cytosol. Recent reports describe substances that inhibit StxB binding to Gb3, but an effective inhibitor of StxA enzymatic activity has not been previously identified.

### SUMMARY

Methods are described for treating a subject suffering from a condition caused by exposure to a toxin, such as an enterotoxin, for example, a Shiga toxin or a cholera toxin. The disclosed methods include enterically administering, such as administering intraluminally, a polyphenolic component of, or a fraction of an extract of the bracts of Humulus luplus (Hops) to neutralize pathogenic bacterial toxins. Administration of the hop component in combination with antibiotics reduces the effect of increased toxin production associated with antibiotic treatement of enterohemorrhagic diseases. Also disclosed are methods and devices for isolating polyphenolic compounds that bind bacterial toxins, and methods and devices for detecting the presence of bacterial toxins in biological samples. Fractions, subfractions and components of hop bract tannin that may be used in any of the disclosed methods and devices also are disclosed.

The foregoing and other features and advantages will become more apparent from the following detailed description of several embodiments, which proceeds with reference to the accompanying figures.

### BRIEF DESCRIPTION OF THE FIGURES

FIGS. 1a-d are bar graphs illustrating the effect of hop bract extract (HBE, FIG. 1a), hop bract tannin (HBT, FIG. 1b), and hop-bract extract low molecular weight fraction (HBE-LMW, FIG. 1c) on RNA N-glycosidase activity of Stx1, the effect of HBT on StxA N-glycosidase activity (FIG. 1d), and the effect of added EDTA on N-glycosidase activity in the presence of HBT.

FIGS. 2a-d are a set of graphs illustrating the effects of HBT on protein synthesis (FIG. 2a) and cell viability (FIGS. 2b, 2c and 2d) for Vero cells in the presence of Stx1.

FIGS. 3a-b are a digital image (FIG. 3a) and a bar graph (FIG. 3b) demonstrating the counteracting effect of HBT on Stx1-induced fluid accumulation in a rabbit ileal loop model.

FIGS. 4a-b are graphs illustrating the kinetics of HBT neutralization of Stx1's effects on protein synthesis in rabbit reticulocyte lysate (raw data, FIG. 4a; Lineweaver-Burke plot, FIG. 4b).

FIGS. 5a-c are a graph, a digital image and a pair of diagrams demonstrating and illustrating the formation of specific HBT-Stx1 complexes. In FIG. 5a, the signal generated using a Biacore sensor having HBT as the bioreceptor demonstrates the specificity of HBT complex formation with Stx1 relative to other proteins. HBT-Stx1 complex formation and precipitation is shown in FIG. 5b. FIGS. 5c and 5d show, respectively, a polyphenolic component of HBT and a model that may explain the behavior observed in FIGS. 5a and 5b.

FIG. 6 is a series of light micrographs (top panels) and fluorescent micrographs (bottom panels) showing binding of fluorescent-labeled Stx1 to a Vero cell surface in the absence of HBT and showing no binding of the labeled Stx1 to Vero cell surfaces in the presence of HBT.

### DETAILED DESCRIPTION

I. Abbreviations

HBT (HBE-HMW)—hop bract tannin [hop bract extract, high molecular weight fraction (Mw>5 kDa) or a polyphenolic component or subfraction thereof].
Stx—Shiga toxin, also known as verotoxin or Shiga-like toxin.
Stx1—Shiga toxin 1.
Stx2—Shiga toxin 2.
StxA—the catalytic A-subunit of a Shiga-toxin.
StxB—membrane binding B-subunit of a Shiga-toxin.
HBE—an extract of hop bracts comprising polyphenolic compounds.
HBE-LMW—hop bract extract, low molecular weight fraction.
STEC—Shiga toxin producing Eschericia coli

II. Terms

Unless otherwise explained, all technical and scientific terms used herein have the same meaning as commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd, 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

The terms “enteric” and “enterically” refer to the gastrointestinal tract, whereas the terms “intraluminal” and “intraluminally” refer specifically to the intestines (small and/or large). The term “enteric administration” refers to delivery of an agent to at least a part of the gastrointestinal tract. For example, enteric administration includes, without limitation, administration through an enteric tube (for example, through an endoscope or plastic tube introduced through the gastrointestinal tract), or in an oral formulation, such as a tablet or liquid. The term “theranostic” refers to a treatment having both a diagnostic and therapeutic component. Theranostic, for example, may refer to a treatment with an agent, where the agent is selected based on the results of a diagnostic test designed to reveal which particular agent is expected to provide the most efficacious treatment. The term “exotoxin” refers to a toxin produced by a microorganism and the term “enterotoxin” refers to a toxin that shows toxicity toward intestinal cells.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. The term “comprises” means “includes.”

All publications, patent applications, patents, and other references mentioned herein are incorporated by reference in their entirety. In case of conflict, the present specification, including explanations of terms, will control. In addition, the materials, methods, and examples are illustrative only and not intended to be limiting. Various embodiments are illustrated by the following non-limiting Examples.

### III. EXAMPLES

Hop bract tannin (HBT) specifically binds toxin molecules, such as cholera toxin, E. coli heat-labile enterotoxin and Stx, and enables methods for treating a subject suffering from an infection caused by a toxin-producing bacteria. These methods include administering to the subject a therapeutically effective amount of hop bract tannin. Administration of HBT may be accompanied by administration of a therapeutically effective amount of an antibiotic that is capable of killing at least a portion of the toxin-producing organisms. The HBT may be administered enterically, such as intraluminally, to block the action of the toxin. Enteric administration includes, without limitation, administration through an enteric tube (for example, through an endoscope or plastic tube introduced through the gastrointestinal tract), or in an oral formulation, such as a tablet or liquid. The oral formulation can be designed to dissolve enterically. In a particular example, the oral formulation is enterically coated to dissolve in a target region of the gastrointestinal tract, for example, the intestines, for example the small intestine or the large intestine. The infection to be treated may present itself clinically as severe diarrhea, hemorrhagic colitis, hemolytic uremic syndrome, thrombotic thrombocytopenic purpura and combinations thereof.

The HBT may be the high molecular weight fraction (≧5 kDa) of a hop bract extract, a particular component thereof, or a subfraction thereof (e.g., a fraction obtained from the high molecular weight fraction of a hop bract extract, and described by an weight-average molecular mass between 5 kDa and 30 kDa). In one embodiment, the HBT comprises a catechin polymer, or a mixture of one or more such polymers, such as a polycatechin selected from the group of 10-mers to 30-mers, and mixtures thereof. In particular embodiments, the polycatechin may have the formula 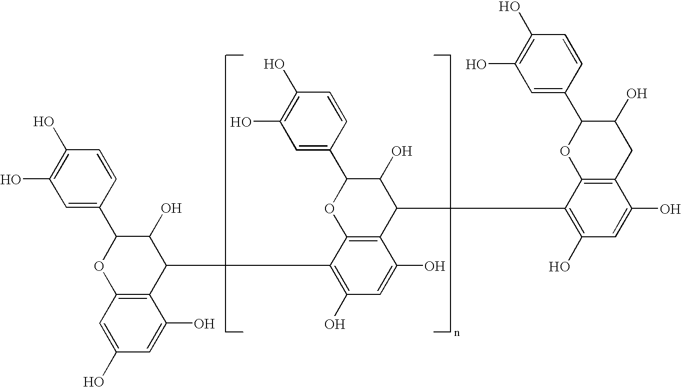
where n=8 to 28. In other particular embodiments, the polycatechin may have the formula 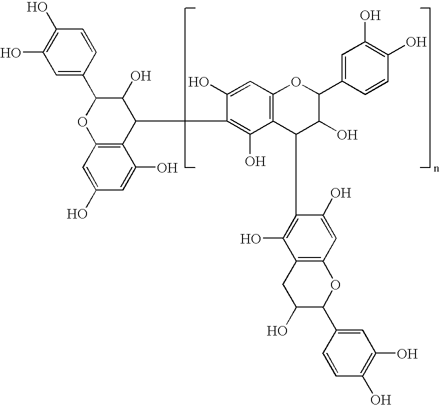
where n=8 to 28.

In yet other particular embodiments, the polycatechin may be a catechin polymer where the linkages between individual catechin molecules are any combination of the linkages shown in the two structures above. For example, if the polycatechin is a 30-mer, there may be anywhere from 1 to 28 linkages of one type, and anywhere from 28 to 1 linkages of the other type.

In other embodiments, the HBT comprises a high molecular weight fraction isolated from hop bract extract (HBE), such as a fraction having a weight-average molecular mass between 5 kDa and 30 kDa.

The Stx-producing organism causing an infection may be an Stx1- or Stx2-producing organism. In particular embodiments the Stx-producing organism is a Shiga toxin-producing Eschericia coli (STEC).

Also disclosed are theranostic methods of treating a subject having an infection caused by an Stx-producing organism. In these methods, a HBT having an affinity for the particular Stx produced by the Stx-producing organism is selected and then administered to the subject enterically, such as intraluminally, in an amount effective to alleviate a clinical presentation of the infection. Desirably, selection is used to identify the HBT fraction or HBT polyphenol that is most effective against the particular Stx produced by the infecting organism.

Selection of the appropriate HBT may be accomplished by affinity chromatography using a chromatographic matrix derivatized with the particular Stx (or combination of Stxs) produced by the infecting organism. Alternatively, selecting the HBT is accomplished simply by obtaining a high molecular weight fraction of a hop bract extract, for example, by selecting a fraction having a weight-average molecular weight of 5 kDa or greater. HBT fractions that most effectively bind the Stx may be more precisely determined, for example, by determining the fractions that precipitate the most Stx (detected visually or electrophoretically).

Particular hop bract polyphenolic compounds, or fractions that have an affinity for the Stx, may also be selected by measuring their affinity for the Stx using a biosensor, where the HBT polyphenol or HBT fraction serves as the bioreceptor portion of the biosensor. Polyphenols that may serve as the bioreceptor include polycatechins, such as between 10-mer and 30-mer polycatechins. HBT fractions may be fractions having a weight-average molecular weight between 5 kDa and 30 kDa. Desirably, the fraction or compounds that provide the greatest signal when used as the biosensor's bioreceptor are selected for administration to the subject.

The specificity of the HBT/Stx interaction also enables methods for detecting the presence of an Stx in a biological sample by contacting the biological sample with hop bract tannin, and detecting a macromolecular complex between the Stx and the hop bract tannin. The macromolecular complex may be detected by observing formation of a precipitate when the biological sample is contacted with the HBT. Complex formation may also be detected by electrophoresis, for example, by observing an electrophoretic pattern associated with the presence of the macromolecular complex in the sample. Alternatively, the HBT may serve as a bioreceptor of a biosensor, and the biosensor may be used to detect the presence of an Stx in a sample. A biosensor comprising an HBT as the bioreceptor and a transducer is also provided.

Methods for isolating and purifying Stx-binding polyphenols are also provided. For example, a mixture comprising an Stx-binding polyphenolic compound isolated from Humulus lupulus may be contacted with Stx to form a macromolecular complex between the compound and Stx. The macromolecular complex may be isolated, and then the polyphenolic compound may be liberated from the macromolecular complex to obtain a purified sample of the polyphenolic compound(s) that bind the Stx. In particular embodiments, the Stx is coupled to an activated chromatographic matrix or a biosensor.

Methods for prophylatic or post-exposure treatment of a condition caused by inhalation of an Stx are also provided. For example, a therapeutically effective amount of HBT may be administered intranasally to a subject to protect the subject from nasal inhalation of the Stx.

The disclosed methods of neutralzing bacterial pathogenicity differ significantly from conventional therapeutic approaches. For example, a vaccine against O157:H7 would not be effective against other STEC serotypes such as O26 and O111. However, methods that utilize HBT (or the components thereof) as Stx-neutralzing agents are effective against diseases caused by all STEC serotypes. HBT may work to prevent intoxication by intraluminal neutralization and elimination of Stx from the body. In contrast, currently available synthetic inhibitors work to block Stx binding to Gb3, leaving the toxin in the body, and therefore available to do damage when the inhibitor concentration drops.

HBT may be derived from abundant natural sources at reduced cost. HBT also exhibits reduced absorption and entrance into the circulatory system. Thus, HBT is more likely to be tolerated by patients, since the effects of HBT would be limited to the alimentary system. Furthermore, since HBT has no effect on bacterial growth, it may be used in combination with other therapeutic modalities, such as antibiotics or transfusion. Continued growth of an organism in the presence of HBT might lead to immunity from extended infection, while the clinical symptoms of intoxication are prevented by the HBT.

### Example 1

### Hop Bract Tannin (HBT)

Hop (Humulus luplus L.) cone is a well-known ingredient in beer, while the hop bract is typically discarded. Hop bract is enriched in highly-condensed catechins (about 50% in polyphenolic fractions). As a by-product of beer brewing, it is available in abundance. Hop bract tannin (HBI) compounds in the high-molecular weight fraction include highly condensed (from about 10-mer to about 30-mer) catechins. Hop bract tannin (HBI) refers to the high molecular weight fraction (≧5 kDa) of a hop bract extract, a polyphenolic component thereof, and mixtures of such polyphenolic components, such as subfractions of the high molecular weight fraction of a hop bract extract comprising one or more such components.

Hop bract samples used for the experiments described in the Examples that follow were prepared by the method of Tagashira et al. (Tagashira et al., “Inhibition by hop bract polyphenols on cellular adherence and water-insoluble glucan synthesis of mutans streptococci,” Biosci. Biotech. Biochem. 61: 332-335, 1997). In brief, an EtOH/H₂O solvent was used to extract the polyphenolic constituents from hop bracts. Other solvent systems (e.g. solvent systems comprising other alcohols (for example, metlanol or isopropyl alcohol), ethers (for example, diethyl ether), ketones (e.g. methyl ethyl ketone), acetonitrile, and mixtures thereof) that extract polyphenolic compounds from hop bracts may be employed to provide HBE. The ¹³C-NMR spectrum of the EtOH/H₂O extract was in good agreement with that of the synthetic catechin-polymer (See, Yoneda et al., “Synthesis of high molecular mass condensed tannin by cationic polymerization of flavan 3,4-carbonate,” J. Chem. Soc., Perkin Trans. 1: 1025-1030, 1997).

Low-(HBE-LMW) and high-molecular weight fractions (HBT) of HBE were separated by ultrafiltration using a 5,000 MW cutoff filter (Amicon Ultra, Millipore, Bedford Mass.). The lower molecular weight limit of the high-molecular weight fraction may be determined by the choice of the filter cutoff and may be anywhere between about 5 kDa and 30 kDa. The higher molecular weight limit of the high molecular weight fraction is typically determined by the molecular weight limit of the components of the HBE itself, but may be lowered by ultrafiltration of the fraction with a second, higher molecular weight cutoff filter and retaining the resulting filtrate as the high molecular weight fraction.

By spectrometric analysis, acidic degradation of HBT in an alcohol solvent yielded only cyanidine; no gallic acid or delphinidine was detected. Gel permeation chromatography (GPC) experiments showed that the HBT had a weight-average molecular mass (Mw) of 6280, a number-average molecular mass (Mn) of 2260, and Mw/Mn=2.8. The TOF-MS spectrum showed regular interval peaks at M/z=288 (native HBT) or M/z=498 (acetylated HBT).

In addition to fractions, individual HBT polyphenolic compounds or mixtures thereof may be isolated from HBE and used as HBT in the disclosed methods. For example, affinity chromatography using an endotoxin derivatized chromatography matrix (See Example 9) may be employed to isolate individual components of HBE. Alternatively, individual components of HBT may be separated and purified using size exclusion HPLC (e.g. Zorbax GF-250 or GF-450 column, Mac-Mod Corp., Chadds Ford, Pa.).

HBT polyphenolic compounds may be described by Formulas 1 or 2 below, where n=8 to 28. In addition, polyphenolic compounds having any combination of the linkages shown in Formulas 1 and 2 may be isolated (i.e. polyphenolic compounds having a mixture of 4→8 linkages as in Formula 1 and 4→6 linkages as in Formula 2). Furthermore, one or more of the OH groups in these structures may be derivatized to form ester and/or ether groups. Esters include, but are not limited to carboxylate (e.g. acetate and propionate), phosphate and sulfate esters. Ether groups include alkoxy groups such as methoxy and ethoxy groups.

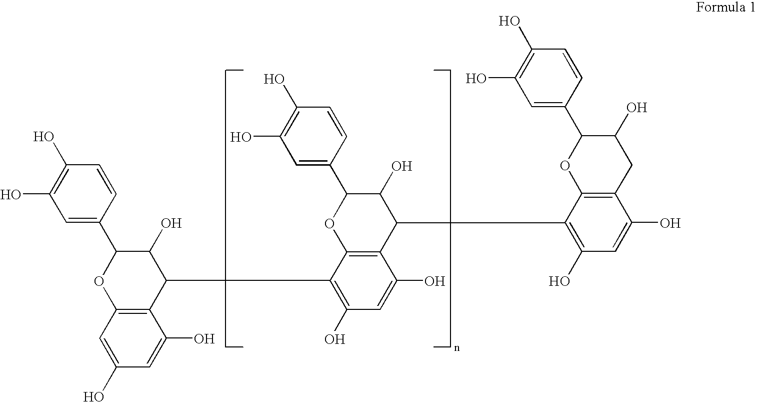

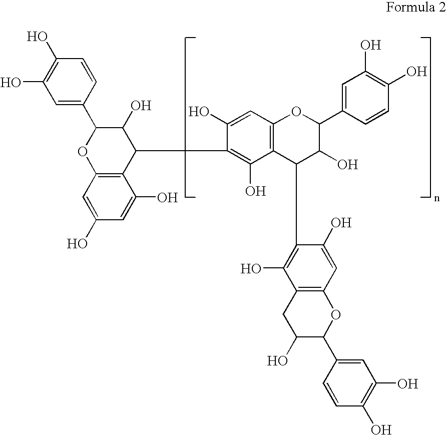

Alternatively, fractions of compounds falling within particular molecular weight ranges may be isolated from hop bract extract (e.g. by ultrafiltration or size exclusion chromatography) and used in the disclosed methods. For ultrafiltration the range of molecular weights depends upon the molecular weight cut-off of the membrane(s) used. For example, fractions containing compounds having weight-average molecular masses in ranges such as 5 kDa-30 kDa, 5 kDa-10 kDa, 5 kDa-8 kDa, 8 kDa-30 kDa, 8 kDa-10 kDa and 10 kDa-30 kDa may be isolated from HBE using commercially available ultrafiltration membranes (e.g. Millipore, Bedford Mass. and Vivascience, Acton Mass.). For size exclusion chromatography, collecting the appropriate fractions as they elute from the column may be used to isolate a fraction having any arbitrary range of molecular weights.

### Example 2

### Exotoxins

The HBT fractions and HBT polyphenols disclosed herein may effectively neutralize a variety of exotoxins, including enterotoxins, such as Shiga toxins and cholera toxins. Cholera toxins are described, for example, in Burrows, “Cholera toxins,” Annu. Rev. Microbiol., 22:245-268, 1968, and include cholera toxins A and B.

As used herein, the terms “Shiga toxin” and “Stx” refer to toxins in the Shiga toxin family that may be neutralized by administration of HBT. The Shiga toxin family contains two types of toxins called Stx1 (verotoxin 1: VT1 or Shiga-like toxin 1: SLT1) and Stx2 (VT2, SLT2), both of which are encoded by bacteriophages. Stx1 resembles the Shiga toxin produced by Shigella dysenteriae type I. Stx2 is heterogeneous. These toxins inhibit protein synthesis in eukaryotic cells, and play a role in hemorrhagic colitis, and hemolytic uremic syndrome. They also have been found to damage endothelial cells in both the kidney and the brain, causing renal failure and neurological complications. (See, for example, Riley et al., New Engl. J. Med., 308: 681-685, 1983 and Ashkenazi, Annu. Rev. Med., 44: 11-18, 1993)

Although many variants exist, all Stx have an A-B structure, where the A-subunit possesses N-glycosidase activity and the B subunit binds to a membrane-bound glycolipid, globotriasoylceramide. The A-polypeptide N-glycosidase activity cleaves an adenine from the 28S rRNA of the 60S cytoplasmic ribosome. This activity renders the 28S rRNA unable to interact with the elongation factors EF-1 and EF-2, thus inhibiting protein synthesis. The B polypeptide forms a pentamer that binds to the eukaryotic cell receptor globotriaosylceramide (Gb₃). Shiga toxins enter cells by receptor-mediated endocytosis. Both Stx1 and Stx2 have both been shown to induce apoptosis in several different cell types.

Stxs have many interesting effects at the cellular level. Once these toxins have been endocytosed, they are transported in a retrograde manner through the Golgi apparatus to the rough endoplasmic reticulum where they effectively target the ribosomes. In addition to inhibiting protein synthesis, Shiga toxins induce production of cytokines such as interleulin-1, interleukin-6, and interleukin-8. They have also been shown to induce expression of tumor necrosis factor (TNF), induce F-actin depolymerization, and activate a src family kinase.

Stx1 is a major virulence factor in the enterohemorrhagic diarrhea caused by Stx-producing Escherichia coli (STEC), such as O157:H7, 89020097 and O157:NM (non-motile). Following administration of antibiotics, E. coli O157:H7 often releases massive amounts of Stx1, resulting in further worsening of symptoms. Other STEC include E. coli within serogroups O26, O103, O111, O113 and O157. Stx2 is also found in STEC. For example, a variant designated Stx(2f) is found in E. coli 0128 (See, Schmidt et al., Appl. Environ. Microbiol., 66:1205-08, 2000).

Stx1 used to demonstrate HBT neutralization of Stx was purified from E. coli MC1061, using pigeon egg ovomucoid-affinity column chromatography according to the method described by Miyake et al. (Miyake et al., “Binding of avian ovomucoid to Shiga-like toxin type 1 and its utilization for receptor analog affinity chromatography,” Anal. Biochem. 281: 202-208, 2000). Purified StxA was obtained by the method of Brigotti et al. (Brigotti et al., “The RNA-N-glycosidase activity of Shiga-like toxin 1:kinetic parameters of the native and activated toxin,” Toxicon 35: 1431-1437, 1997).

### Example 3

### Hop Bract Tannins Inhibit RNA N-glycosidase Activity of Stx1

This example demonstrates that hop bract tannin (HBT) inhibits the RNA N-glycosidase activity of Stx1. RNA N-glycosidase activity was assayed in a cell-free rabbit reticulocyte system according methods described by Miyake et al. and Sargiacomo et al. (See, Miyake et al., “Binding of avian ovomucoid to Shiga-like toxin type 1 and its utilization for receptor analog affinity chromatography,” Anal. Biochem. 281: 202-208, 2000 and Sargiacomo et al., “Cytotoxicity acquired by ribosome-inactivating proteins carried by reconstituted Sendai virus envelopes,” FEBS Lett. 157: 150-154, 1983). Rabbit reticulocyte lysate was prepared from female rabbits (New Zealand White, 3 kg, Japan SLC, Japan).

Rabbit reticulocyte lysate and samples (Stx1/HBT) dissolved in PBS were mixed at 4° C. (total 50 μl). After additions of 20 μl of reaction mixture (described below), and incubation at 30° C. for 0-15 min, as indicated, 1 ml of 10% TCA was added and samples were boiled in 95° C. water bath for 10 min. Precipitates were collected on filters and washed with 3 ml of 10% TCA, before radioassay of [¹⁴C]. Reaction mixtures were prepared from 36.6 ml of rabbit reticulocyte lysate and contained 15 mM HEPES (pH 7.5), 1 mM ATP, 0.2 mM GTP, 15 mM phosphocreatine, 150 ug/ml creatine kinase, 2 mM magnesium acetate, 66 mM KCl, 6 mM dithiothreitol, 240 ug/ml haemin, 0.1 mM of each of 19 amino acids (no leucine), and 6.8 uCi/ml [¹⁴C] leucine (DuPont NEN Research Products, Boston, Mass.).

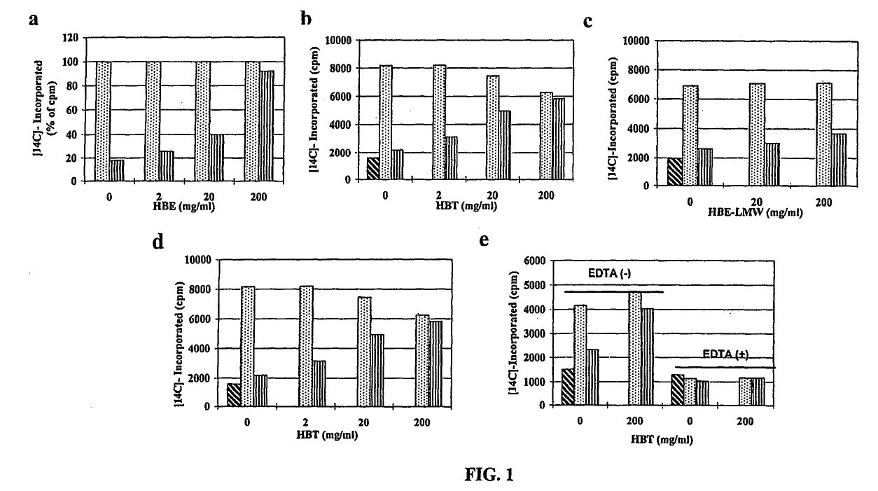

FIG. 1 shows how several different hop bract samples affect the reduction of protein synthesis caused by added Stx1 or StxA. Columns show protein synthesis in rabbit reticulocyte lysate without 37° C. incubation (cross-hatched, negative control), at 37° C. without Stx1 (dots, positive control); and in the presence of either Stx1 or StxA (vertical lines). Column height shows mean±SD [¹⁴C] radioactivity (n=3) incorporated by the sample due to protein synthesis.

In the rabbit reticulocyte lysate system, natural source hop bract extract (HBE) inhibited the RNA N-glycosidase activity of Stx1 in a dose dependent fashion. Addition of HBE increased protein synthesis by counteracting the effects of Stx1, and, at a concentration of 200 μg/mL, returned protein synthesis to levels similar to that seen without Stx1 (FIG. 1a). The high-molecular weight fraction of HBE (HBT) alone was also a potent inhibitor of Stx1 activities (FIG. 1b) and restored protein synthesis in a dose-dependent fashion. To the contrary, the low-molecular weight fraction (HBE-LMW) had little inhibitory effect on Stx1 activity (FIG. 1c). HBT inhibited both Stx (FIG. 1b) and purified StxA (FIG. 1d), suggesting that HBT binds directly to the A-subunit of Stx1. When EDTA inhibited total protein synthesis, HBT did not increase radioactivity on the filter (FIG. 1e), indicating that the increase in the presence of HBT (FIGS. 1b, d) was not caused by non-specific capture of [¹⁴C] leucine by HBT.

### Example 4

### HBT Inhibits Cytotoxicity of Stx1 Toward Vero Cells

This example demonstrates that HBT is effective for reducing the toxic effects of Stx1 on kidney cells. Vero cells were seeded in a 96-well microtitre plate (2×1 0⁴ cells in 100 μl per well) and grown in minimum essential medium (MEM, Sigma-Aldrich, St. Louis, Mo.) containing 10% fetal bovine serum (FBS, JRH Biosciences, Lenexa, Kans.), at 37° C. in a 5% CO₂ atmosphere. Confluent cell monolayers were used for the assays. Vero cells were seeded approximately 2×10⁵ cells (in 1 ml) in each well of a 24-well microtitre plate and cultured for 48 h. The plate was cooled on ice for 10 min and then the medium was replaced with 0.5 ml of MEM-10% FBS, containing 0.9 uCi/Ml of [¹⁴C] leucine. After addition of Stx1 and/or HBT (in 50 μl), the plate was incubated at 37° C. for 40 min on a water bath. Protein synthesis was stopped by addition of 0.25 ml of 30% TCA. Cells were washed three times with 1 ml of 10% TCA and lysed in 0.25 ml of 0.5 N KOH for 10 min at 37° C. The lysate was neutralized with 0.25 ml of 0.5 N acetate and protein synthesis was quantified by radioassay of [¹⁴C].

Several concentrations of HBT and Stx1 were diluted in PBS solution and mixed (80 μl final per well) in another 96-well microtitre plate. The plate was incubated for 1 h at 37° C., after which 10 μl from each well were added to wells containing Vero cells. The Vero cell plate was incubated for an additional 48 h at 37° C. in a 5% CO₂ environment. The viability of Vero cells was measured by Cell Counting Kit (Dojindo Laboratories, Kumamoto, Japan), according to the MTT-assay method (Roche Diagnostics Corporation, Indianapolis, Ind.). The MTT method is based on spectrophotometric detection of the cleavage of a tetrazolium salt by a mitochondrial respiratory chain enzyme, and is a measure of metabolic activity and cell viability.

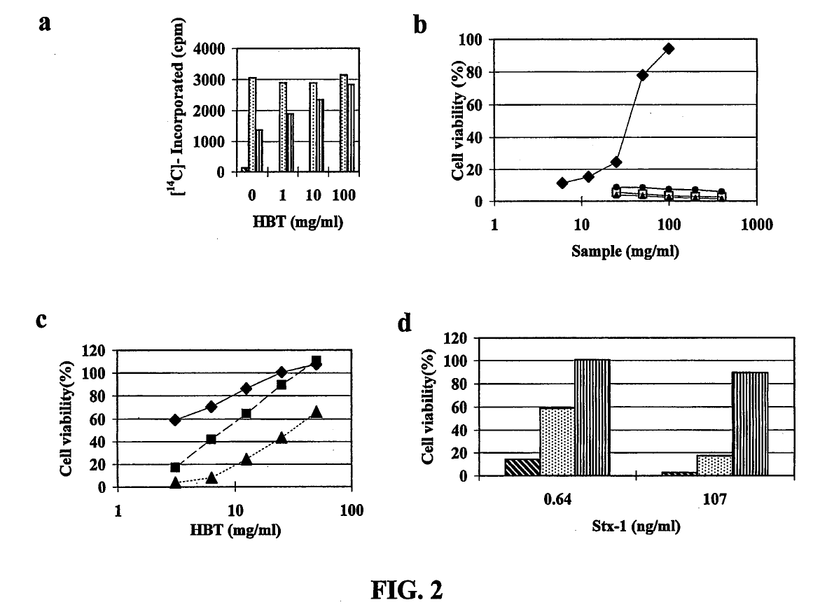

Stx1 modifies ribosomal RNA irreversibly, thereby inhibiting protein synthesis and causing cell death. HBT protected Vero cells, in a dose-dependent manner, from inhibition of protein synthesis during a 45-min exposure to Stx1 (FIG. 2a). The columns in FIG. 2a show the mean±SD of [¹⁴C] radioactivity (n=3) incorporated by Vero cells in the presence of varying amounts of HBT, without 37° C. incubation (cross-hatched, negative control), without Stx1 (dots, positive control) and in the presence of with Stx1 (0.7 mg/ml) (vertical lines). These results are consistent with the results shown in Example 3 for the rabbit reticulocyte lysate assay.

The effect of HBT and other polyphenol samples on the viability of Vero cells was also investigated. FIG. 2b shows the mean±SD of MTT-assay data (n=8) for Vero cells that were incubated with Stx1 (62 μg/ml) at 37° C. for 2 days in the presence of HBT (diamonds), HBE-LMW (squares), green tea polyphenol (GTP, triangles), and oolong tea polyphenol (OTP, circles). FIG. 2b demonstrates that HBE-LMW, GTP and OTP fractions did not have protective effects on Vero cells in the presence of Stx1, whereas HBT prevented cell death under similar experimental conditions.

The protection afforded by HBT against Stx1 toxicity at varying concentrations was also investigated. With reference to FIG. 2c, cells were treated with Stx1 at three different concentrations [0.64 ng/ml (diamonds), 107 ng/ml (squares), 227 ng/ml (triangles)]. For this study the Vero cells were exposed to Stx1 and HBT at 4° C. (on ice) for 30 min, washed with PBS for twice and incubated at 37° C. for 2 days in MEM-10% FBS. The results show that the protective effect (increased cell viability) of HBT depends on Stx1 concentration and time of exposure of Vero cells to Stx1 (FIG. 2). HBT was more effective in neutralizing Stx1 activity during short exposure times. For longer incubation times, residual free Stx1 may bind to Vero cells.

FIG. 2d presents data similar to that shown in FIG. 2c and further demonstrates the protective effect of HBT. Specifically, FIG. 2d shows the effect of HBT on cell viability in the presence of Stx1 at two concentrations under the experimental conditions used to generate FIG. 2d. With reference to FIG. 2d, the relative cell viability in the presence of Stx1 alone (cross-hatches) and in the presence of both Stx-1 and HBT at 3.1 μg/mL (dots) or 25 μg/mL is shown.

### Example 5

### HBT Inhibits Stx1-Induced Fluid Accumulation in Rabbit Ileal Loops

This example illustrates how HBT neutralizes Stx1 action on cells in the mammalian intestine and demonstrates the utility of HBT for preventing Stx1-induced diarrhea. Fluid accumulation in rabbit ileal loops induced by Stx1 was evaluated using the methods described by St. Hilaire et al. (St. Hilaire et al., “Interaction of Shiga-like toxin type 1 B-subunit with its carbohydrate receptor,” Biochemistry, 33: 14452-14463, 1994). Male rabbits (Japanese white, 2 kg purchased from Japan SLC) were starved for 48 hr before operation, although water was available ad libitum. Rabbits were anesthetized with thiopental sodium and the intestine was exteriorized through a midline incision. In each rabbit, 6-10 segments (about 6-8 cm in length) were isolated and 100 ng of Stx1 and/or HBT sample (total volume 1 ml) were simultaneously injected into each loop. Rabbits were sacrificed 24 hr. later, and the loops excised. The ratio of the volume of accumulated fluid within the loop per the length of the loop (ml/cm) is the measure of Stx1 toxic activity.

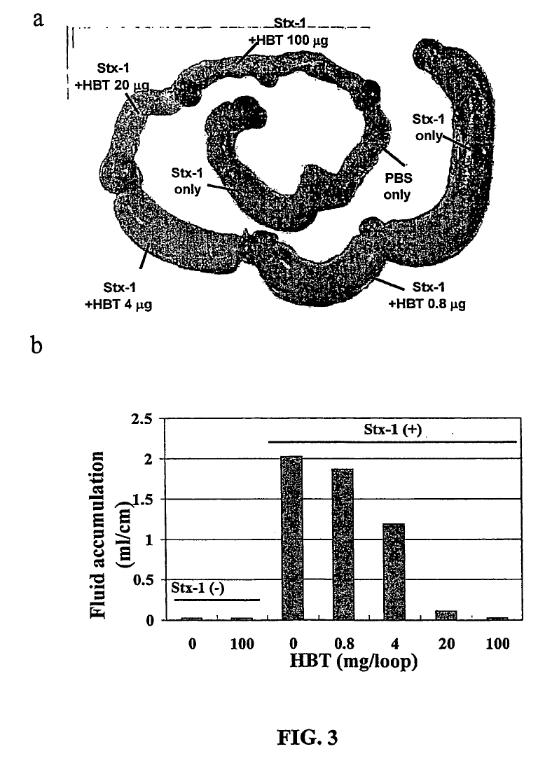

HBT showed potent, dose-dependent inhibition of fluid accumulation induced by Stx1 (FIGS. 3a and 3b). As can be clearly seen in FIG. 3a, severe swelling of the intestine is induced by Stx1. Co-administration of HBT and Stx1 leads to reduced amounts of swelling, and the appearance of the intestinal segment receiving 100 μg HBT is similar to the control segment receiving only phosphate buffered saline (PBS). These results are quantified in FIG. 3b and it is evident that even low doses of HBT, such as 0.8 μg per loop, provide a measurable reduction in Stx1 induced swelling. These data demonstrate that HBT may be used as an effective therapeutic agent against enterohemorrhagic diarrhea caused by STEC and other exotoxin producing organisms.

### Example 6

### HBT Interferes with Binding of Stx1 to rRNA

To elucidate the mechanism of the toxin-neutralizing action of HBT, a kinetic analysis of the effect of HBT on Stx1 activity was performed by measuring the rate of protein synthesis in a rabbit reticulocyte lysate system.

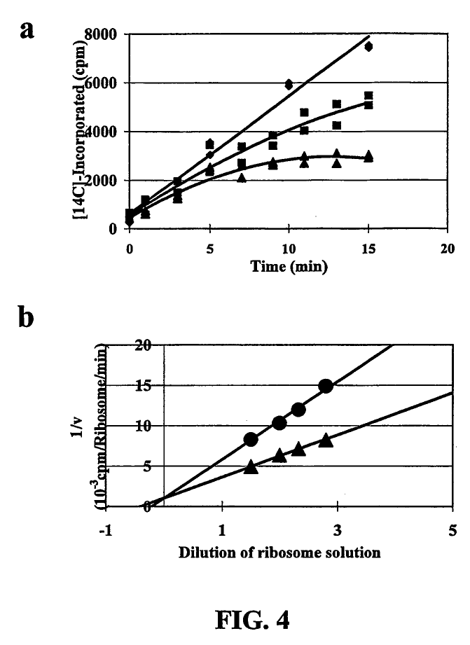

At low concentrations of Stx1 (e.g. 0.7 μg/ml), HBT (3.5 μg/ml) reduced the magnitude of Stx1 inhibition of protein synthesis. FIG. 3a shows the time course plot of the increase in [¹⁴C] radioactivity due to protein synthesis for rabbit reticulocyte lysate without HBT or Stx1 (diamonds), with HBT (3.5 μg/ml) and Stx1 (0.7 μg/ml) (squares); and with Stx1 alone (0.7 μg/ml). Lineweaver-Burk analysis (FIG. 4b) of the kinetic data showed that the inhibition was competitive (i.e. the maximal velocity (Vm) of the Stx1-catalyzed reaction was not changed by HBT, while the Km was increased significantly). Without being bound to a particular theory, these results seem to show that HBT binds to StxA first, and that HBT-Stx1 complex formation prevents StxA binding to ribosomal RNA. In contrast to these findings, some nucleic acid analogues have been reported to inhibit ribosome-inactivating proteins (RIP), including Stx1, noncompetitively (See, for example, Pallance et al., “Uncompetitive inhibition by adenine of the RNA-N-glycosidase activity of ribosome-inactivating proteins,” Biochim. Biophys. Acta 1384: 277-284, 1998; Brigotti, M. et al. 4-Aminopyrazolo [3,4-d]pyrimidine (4-APP) as a novel inhibitor of the RNA and DNA depurination induced by Shiga toxin 1,” Nucleic Acids Res., 28: 2383-2388, 2000; and Brigotti et al., “A survey of adenine and 4-Aminopyrazolo [3,4-d]pyrimidine (4-APP) as inhibitors of ribosome-inactivating proteins (RIPs),” Life Sci. 68: 331-336, 2000).

### Example 7

### HBT Binds Stx1 and Forms a Macromolecular Complex

In general, polyphenols bind to proteins nonselectively (See, for example, Haslam, “Natural polyphenols (vegetable tannins) as drugs: possible modes of action,” J. Nat. Prod., 59: 205-215, 1996). Surprisingly, however, HBT binds Stx1 more avidly than other proteins. HBT binding to several proteins (Stx1, bovine serum albumin, ovalbmin) was compared. Binding between HBT and the proteins was quantified using a Biacore-2000 system (Biacore, Co., Stockholm, Sweden). HBT was non-covalently immobilized on the CA5 or SA sensor tip by repetitive flow of HBT at 50 μg/ml (20 μl/min for 180 sec.). Non-immobilized HBT was washed from the sensor with 0.1 N NaOH (20 μl/min for 60 sec) and the sensor tip was washed with running buffer for 18 h before used for the experiment. Proteins at a concentration of 4.2 nM were mixed with the sensor tip and the change of reaction unit (RU) was measured.

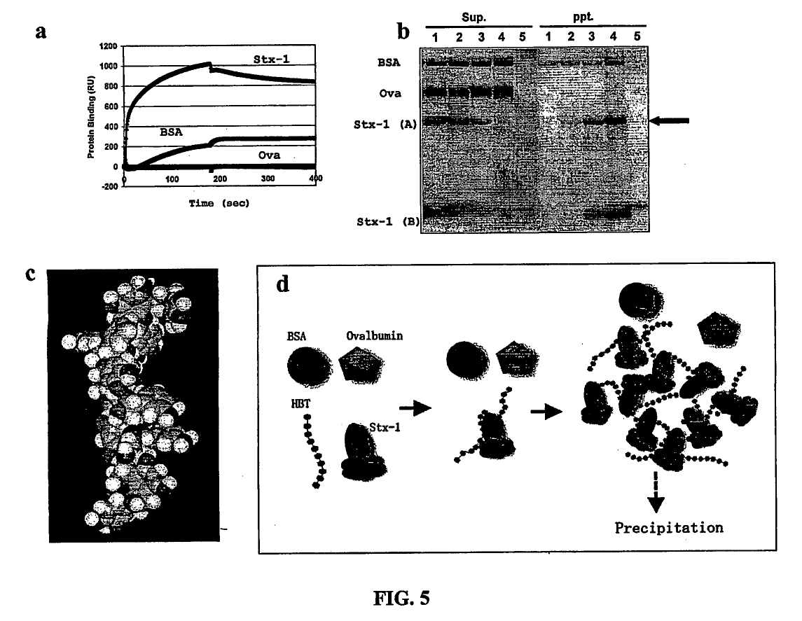

Surprisingly, HBT bound Stx1 more tightly than other proteins (FIG. 5a). Furthermore, following incubation for 1 h at 37° C. in PBS, HBT selectively formed large aggregates with Stx1. These aggregates could be precipitated by centrifugation. Precipitation of HBT-Stx1 complexes was monitored by SDS-PAGE. HBT and 4.2 nM of each of the proteins (i.e, Stx1, BSA, ovalbumin) were mixed (total volume 60 μl) and incubated at 37° C. for 60 min. After centrifugation (60 min, 10,500 g), the supernatant was collected and the tube was gently washed twice with PBS (60 μl). The proteins in the supernatant and the precipitate were visualized after SDS-PAGE by silver-staining (FIG. 5b). A possible explanation is that HBT, with its elongated, bulky structure (shown in FIG. 5c), binds to StxA, leading to formation of large HBT-Stx1 complexes as depicted in (FIG. 5d).

### Example 8

### Inhibition of Stx1 Adherence to Cells

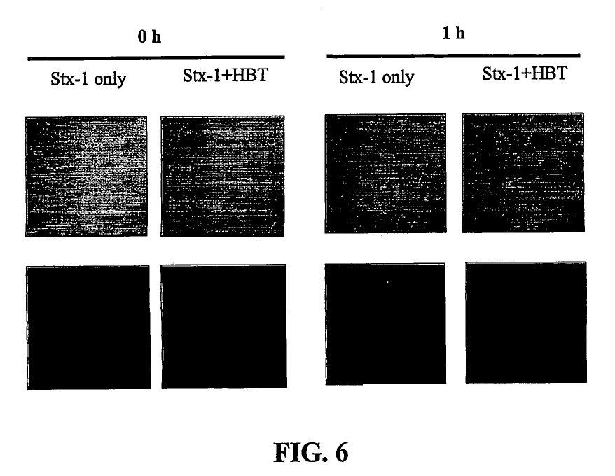

This example demonstrates not only that HBT forms complexes with Stx, it also inhibits Stx binding to Vero cell surfaces. Stx1 was fluorescent-labeled according to the protocol given with the FluoroLink-Ab Cy-3 labeling kit PA 33000 (Amersham Pharmacia Biotech, Uppsala, Sweden). Vero cells were grown on a poly-1-lysine-coated on cover glasses (18×18 mm, Iwaki glass Co., Japan) in a small culture dish. The solution of Cy-3 labeled Stx1 was added to the Vero cell monolayer at 4° C. for 30 min (See, for example, Hitotsubashi et al., “Some properties of purified Escherichia coli heat-stable enterotoxin II,” Infect. Immun., 60: 4468-4474, 1994). After incubation at 37° C. for a period of time (0-1 h), the cells on the cover glasses were washed twice with PBS, and fixed in 3% formaldehyde for 20 min at room temperature. Cells were inspected using a fluorescence-microscope system (Nikon Co., Tokyo, Japan). Visible and fluorescence micrographs of the Vero cells on the cover glasses following brief (0 h) and extended exposure (1 h) to Stx with and without HBT are shown in FIG. 6.

The data clearly demonstrate that HBT inhibits Stx1 binding to the Vero cell surface (after a 30-min exposure to Stx1 at 4° C.), thereby preventing Stx1 translocation to the cytosol (after 60 min at 37° C.). The data also support a conclusion that HBT forms complexes with Stx1, preventing its toxic effects on cells, perhaps through its interaction with the A subunit. The photographs in upper row of FIG. 6 show Vero cells visualized by phase microscopy. In the lower row of FIG. 6, the photographs show Vero cells visualized with fluorescent pigment (Cy-3) labeled Stx1 from the same view. The photographs under bar marked 0 h show the cells after 30 min treatment (at 4° C.) with Cy-3 labeled Stx1, and those under the bar marked 1 h show the cells after 1.0 h incubation at 37° C., following the 4° C., 30 min exposure. For both times, only the cells treated with Stx1 alone show the presence of the fluorescent-labeled Stx-molecules on the surface of the Vero cells. In contrast, addition of HBT prevented adherence of the fluorescent-labeled Stx1 molecules to the Vero cells, as evidenced by the dark fluorescence images.

### Example 9

### HBT has No Antibiotic Activity on O157:H7 and Does Not Interfere with the Action of Antibiotics

This example demonstrates that HBT acts upon the toxin produced by enterohemorrhagic bacteria, rather than the bacteria themselves. E. coli O157:H7, isolated from a male patient in Chiba prefecture, Japan in 1999 (Dr. F. Nomura, Chiba University, Graduate School of Medicine) and cultured in Muller-Hinton Broth medium (Gibco BRL, Grand Island, N.Y., 100 μl), were combined with HBT and/or streptomycin (Meiji Seika, Co. Ltd., Tokyo, Japan) and dissolved in PBS (total volume 10 μl). Samples were added to 96-well plates and incubated overnight (16-20 h) at 37° C. Growth of O157:H7 was measured by absorbance at 600 nm.

Addition of HBT (up to 200 g/ml) had no effect on O157:H7 growth. It also did not prevent the antibiotic effect of streptomycin (data not shown). These results demonstrate that HBT may be administered in combination with antibiotics to provide a treatment directed both toward the organisms themselves and the toxins they produce.

### Example 10

### Isolation of HBT Constituents Effective Against Stx1

The specific interaction between HBT constituents and the Stx1 protein that was demonstrated in Example 7 above may be exploited to isolate polyphenolic components from crude hop bract extract and polyphenolic components from other plant materials. For example, affinity chromatographic methods for isolating polyphenolic compounds based on the specific HBT/Stx interaction are enabled, as is selective precipitation of polyphenolic compounds.

An affinity chromatographic stationary phase is produced by reacting Stx1 molecules with an activated chromatography matrix. Activated matrices of several types are available from Sigma, St Louis, Mo. Preparation of affinity chromatography matricies is described in Boyer, “Modern Experimental Biochemistry,” 2^{nd} Ed., Benjamin/Cummgs Publishing Co, Redwood City, Calif., 1993. For example, cyanogen bromide activated matrices are especially useful for providing Stx-derivatized affinity matrices because all ligands containing primary amino groups (e.g. proteins) are easily attached to cyanogen bromide under mild conditions.

Once prepared, the affinity matrix is placed in a column according to methods well known in the art and a sample, presumably containing polyphenolic compounds capable of interacting specifically with Stx1, is passed through the column. The column is then rinsed to remove weakly bound constituents of the sample. The strongly bound constituents are then eluted from the column using, for example, a solution containing an Stx1 specific antibody or a solution containing a chaotropic agent such as urea, or guanidine.

In one embodiment, crude HBE is passed through an affinity column containing an Stx1 functionalized matrix and the components of the HBE that specifically bind to the matrix are eluted to provide a purified sample of HBT that may be administered to a subject to aggregate Stx1 intraluminally.

### Example 11

### Detection of Enterohemorrhagic Infection

The toxin specific binding properties of the components of HBT enable biosensors and methods for detecting the presence of toxins in biological samples (e.g. blood, urine, feces, or tissue). For example, HBT, or a polyphenolic compound isolated therefrom, is immobilized on a transducer, such as an electrode surface, to provide a bacterial toxin specific sensor. A biological sample, presumably containing the toxin, may then be contacted with the sensor and a change in a property of the transducer may be detected (e.g., a change in the potential or current passing through an electrode). Sensor response may be calibrated against standard solutions of the toxin and used to quantify the amount of the toxin in the biological sample.

A biosensor includes a biological recognition system (bioreceptor) and a transducer. The interaction of the analyte with the bioreceptor produces an effect measured by the transducer that may be converted, for example, into an electrical signal. Transducer types include optical transducers (e.g. luminescence, absorption, surface plasmon resonance), electrochemical transducers and mass-sensitive transducers (e.g. surface acoustic waves, microbalances). Optical transducers may be based on different types of spectroscopy (e.g. absorption, fluorescence, phosphorescence, Raman, SERS, refraction or dispersion) and different spectrochemical properties may be monitored (e.g. amplitude, energy, polarization, decay time and/or phase). Electrochemical transducers include conducting polymers (e.g. poly N-methylpyrroles, polyanilines, and poly ophenylenediamine), carbon and metals (e.g. gold and platinum). Mass-sensitive transducers include piezoelectric crystals. The bioreceptor may be attached to the transducer either covalently or non-covalently. Additional details of biosensor technology are described by Vo-Dinh and Cullum (Vo-Dinh and Cullum, “Biosensors and biochips: advances in biological and medical diagnostics,” Frsenius J. Anal. Chem., 366:540-551, 2000).

In a particular embodiment, a microarray of biosensors is provided. These “biochips” may include particular polyphenolic HBT compounds or fractions (e.g. 10-mer through 30-mer polycatechins or fractions having particular mass ranges or average masses) deposited on individual transducer elements to form an array of detectors. Such biochips are useful for determining the most effective treatment for a particular toxin-mediated infection (i.e. theranostic determinations). For example, a sample of the toxin produced by a microorganism may be contacted to a biochip having polyphenolic compounds as bioreceptors and the polyphenolic compound that most effectively binds the toxin is identified by the transduced signal it produces relative to the other polyphenolic compounds serving as bioreceptors on the biochip. Once identified, the strongest binding polyphenolic compound may be administered to a subject. For example, a subject may ingest the compound to intraluminally precipitate the toxin and increase its elimination, while simultaneously attenuating the toxin's effects on intestinal cells.

The Biacore system used above in Example 7 is an example of a biosensor that incorporates immobilized HBT compounds as bioreceptors. The Biacore sensor chip transducer operates by surface plasmon resonance. If sufficient amounts of protein can be recovered from the surface of such chips, it may be possible to identify ligands using mass spectrometry. For example, proteins may be vaporized using matrix assisted laser desorption directly from the sensor surface or proteins eluted from the sensor surface may be measured following electrospray ionization.

In another embodiment, a biological sample is contacted with a solution containing HBT and any precipitate formed due to the formation of HBT-toxin macromolecular complexes is separated from the resulting solution by, for example, centrifugation. In one embodiment, the presence of Stx1 in the sample is indicated by the presence of a precipitate. In a more particular embodiment, the amount of Stx1 in the biological sample is quantified by measuring, such as by gravimetric analysis, the amount of precipitate. Calibration standards may be employed.

### Example 11

### Detection of Microbial Toxin Aerosols

The sensors described in Example 10 above may also be used to detect the presence of microbial toxins in an enviroment. For example, a biosensor having HBT components as the bioreceptor may be used to detect the presence of microbial toxins in the air or on surfaces. Presence of microbial toxins may be detected by contacting the sensor with, for example, a solution prepared from a filtered air sample or a solution prepared from a swab sample of a surface. Such sensors may find utility as early-warning detectors of attacks with microbial toxins.

### Example 12

### Protection from Microbial Toxin Aerosols

Because they effectively form complexes and selectively precipitate microbial toxins, especially Shiga toxins, HBTs may be administered either prophylactically or post-exposure to prevent development of the symptoms of intoxication (e.g. in an aerosol, drinking water, or food). In a particular embodiment, HBT, or one or more components thereof, are administered intranasally to precipitate and neutralize Shiga toxins that have been or might be inhaled by a subject.

### Example 13

### Pharmaceutical Compositions

Pharmaceutical formulations according to the present invention encompass formulations that include an amount (for example, a unit dosage) of a toxin neutralizing agent together with one or more non-toxic pharmaceutically acceptable excipients, including carriers, diluents, and/or adjuvants, and optionally other biologically active ingredients such as a therapeutically effective amount of an antibiotic where the amount can kill at least a portion of a pathogen population. Standard pharmaceutical formulation techniques are used, such as those disclosed in Remington's Pharmaceutical Sciences, Mack Publishing Co., Easton, Pa. (19th Edition).

A pharmaceutical formulation according to the invention includes HBT fractions and/or one or more purified HBT polyphenols, and can also include, for example, one or more other biologically active ingredients, such as cefixime, tetracycline, ciprofloxacin, co-trimoxazole, norfloxacin, ofloxacin, fosfomycin and kanamycin and combinations thereof.

The dosage of the combined biologically active agents is sufficient to achieve concentrations at the site of action that are similar to those that are shown to achieve in vivo protection from microbial toxins. Pharmaceutical formulations may include, for example, an amount of a toxin-neutralizing agent such that the subject receives a dosage of between about 0.000 μg/kg and 100 g/kg.

The compositions can be in the form of tablets, capsules, powders, granules, lozenges, liquid or gel preparations, such as oral, topical, or solutions or suspensions (e.g., eye or ear drops, throat or nasal sprays, etc.) and other forms known in the art.

Such pharmaceutical compositions can be administered systemically or locally in any manner appropriate to the treatment of a given condition, including orally, rectally, nasally, buccally, by inhalation spray, or via an implanted reservoir.

Pharmaceutically acceptable carriers include, but are not limited to, ion exchangers, alumina, aluminum stearate, lecithin, serum proteins (such as human serum albumin), buffers (such as phosphates), glycine, sorbic acid, potassium sorbate, partial glyceride mixtures of saturated vegetable fatty acids, water, salts or electrolytes such as protaine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Tablets and capsules for oral administration can be in a form suitable for unit dose presentation and can contain conventional pharmaceutically acceptable excipients. Examples of these include binding agents such as syrup, acacia, gelatin, sorbitol, tragacanth, and polyvinylpyrrolidone; fillers such as lactose, sugar, corn starch, calcium phosphate, sorbitol, or glycine; tableting lubricants, such as magnesium stearate, talc, polyethylene glycol, or silica; disintegrants, such as potato starch; and dispersing or wetting agents, such as sodium lauryl sulfate. Oral liquid preparations can be in the form of, for example, aqueous or oily suspensions, solutions, emulsions, syrups or elixirs, or can be presented as a dry product for reconstitution with water or other suitable vehicle before use.

The pharmaceutical compositions can also be administered enterally in a sterile aqueous or oleaginous medium. The composition can be dissolved or suspended in a non-toxic enterally-acceptable diluent or solvent, e.g., as a solution in 1,3-butanediol. Commonly used vehicles and solvents include water, physiological saline, Hank's solution, Ringer's solution, and sterile, fixed oils, including synthetic mono- or di-glycerides, etc. Additives may also be included, e.g., buffers such as sodium metabisulphite or disodium edeate; preservatives such as bactericidal and fungicidal agents, including phenyl mercuric acetate or nitrate, benzalkonium chloride or chlorhexidine, and thickening agents, such as hypromellose.

The dosage unit involved depends, for example, on the condition treated, nature of the formulation, nature of the condition, embodiment of the claimed pharmaceutical compositions, mode of administration, and condition and weight of the patient. Dosage levels are typically sufficient to achieve a tissue concentration at the site of action that is at least the same as a concentration that has been shown to neutralize microbial toxins in vitro. For example, a dosage of about 0.0001 g/kg and 100 g/kg of the active ingredient may be useful in the treatment of toxin-mediated conditions. The unit dosage can also be formulated to include both the HBT and another therapeutic agent, such as an anti-infective agent, for example, an antibiotic.

The compounds can be used in the form of salts, preferably derived from inorganic or organic acids and bases, including, but not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, perulfate, 3-phenylpropionate, picrate, pivalate, propionate, succinate, tartrate, thiocyanate, tosylate, and undecanoate. Base salts include, but are not limited to, ammonium salts, alkali metal salts (such as sodium and potassium salts), alkaline earth metal salts (such as calcium and magnesium salts), salts with organic bases (such as dicyclohexylamine salts), N-methyl-D-glucamine, and salts with amino acids (such as arginine, lysine, etc.). Basic nitrogen-containing groups can be quaternized, e.g., with such agents as C1-8 alkyl halides (such as methyl, ethyl, propyl, and butyl chlorides, bromides, and iodides), dialkyl sulfates (such as dimethyl, diethyl, dibutyl, an diamyl sulfates), long-chain halides (such as decyl, lauryl, myristyl, and stearyl chlorides, bromides, and iodides), aralkyl halides (such as benzyl and phenethyl bromides), etc. Water or oil-soluble or dispersible products are produced thereby.

Pharmaceutical compositions can be included in a kit accompanied by instructions for intended use, for example instructions required by a pharmaceutical regulatory agency, such as the Food and Drug Administration in the United States.

It will be apparent that the precise details of the methods or compositions described may be varied or modified without departing from the spirit of the described invention. We claim all such modifications and variations that fall within the scope and spirit of the claims below.

1. A method for treating a subject having an infection caused by an Stx-producing organism by administering to the subject a therapeutically effective amount of hop bract tannin.

2. The method of claim 1 further comprising administering to the subject a therapeutically effective amount of an antibiotic, the antibiotic being effective to treat an infection with the Stx-producing organism.

3. The method of claim 2, wherein the antibiotic is selected from the group consisting of cefixime, tetracycline, ciprofloxacin, co-trimoxazole, norfloxacin, ofloxacin, fosfomycin and kanamycin and combinations thereof.

4. The method of claim 1, wherein the hop bract tannin comprises a catechin polymer.

5. The method of claim 4, wherein the catechin polymer comprises a polycatechin between a 10-mer and a 30-mer.

6. The method of claim 1, wherein the infection is an enteric infection.

7. The method of claim 6, wherein the hop bract tannin is administered enterically.

8. The method of claim 5 where the polycatechin has the formula 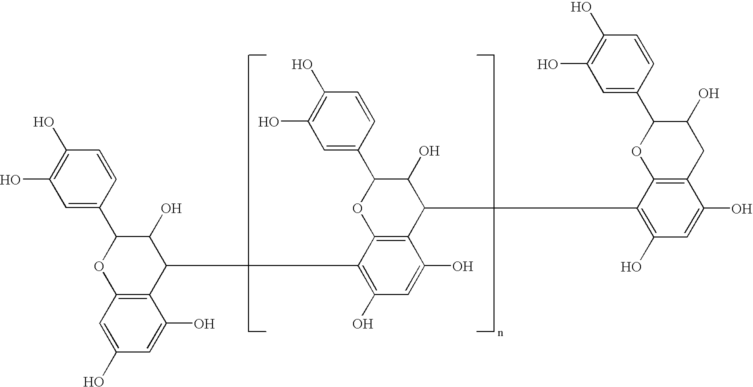
where n=8 to 28.

9. The method of claim 5 where the polycatechin has the formula 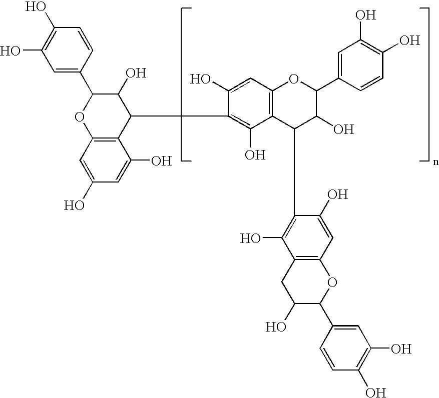
where n=8 to 28.

10. The method of claim 1, wherein the hop bract tannin comprises a fraction isolated from a hop bract extract.

11. The method of claim 10, wherein the fraction has a weight-average molecular mass between 5 kDa and 30 kDa.

12. The method of claim 1, wherein the Stx-producing organism comprises an Stx1-producing organism.

13. The method of claim 1, wherein the Stx-producing organism is a Shiga toxin-producing Eschericia coli.

14. The method of claim 1, wherein the infection is an enteric infection, and the hop bract tannin comprises a polycatechin between a 10-mer and a 30-mer, which is administered enterically.

15. The method of claim 14, wherein the infection presents clinically as severe diarrhea, hemorrhagic colitis, hemolytic uremic syndrome and thrombotic thrombocytopenic purpura.

16. (canceled)

17. The method of claim 1, wherein administering to the subject a therapeutically effective amount of hop bract tannin comprises:
selecting a hop bract tannin having an affinity for an Stx produced by the Stx-producing organism; and
administering the hop bract tannin to the subject enterically in an amount effective to alleviate a clinical presentation of the infection.

18. The method of claim 17, wherein selecting comprises isolating hop bract tannin from a hop bract extract by affinity chromatography with a chromatographic matrix derivatized with the Stx.

19. The method of claim 17, wherein selecting comprises obtaining a high molecular weight fraction of a hop bract extract.

20. The method of claim 19, wherein the high molecular weight fraction has a weight-average molecular weight of 5 kDa or greater.

21. The method of claim 17, wherein selecting comprises detecting a hop bract tannin component having an affinity for the Stx.

22. The method of claim 21, wherein detecting a component having an affinity for the Stx comprises detecting a signal generated by a biosensor, the biosensor having a hop bract tannin as a bioreceptor portion of the biosensor.

23. The method of claim 22 where the hop bract tannin is a polycatechin.

24. The method of claim 23 where the polycatechin is between a 10-mer and a 30-mer polycatechin.

25-26. (canceled)

27. The method of claim 17, wherein the clinical presentation of the infection is one or more of severe diarrhea, hemorrhagic colitis, hemolytic uremic syndrome and thrombotic thrombocytopenic purpura.

28. (canceled)

29. A method for detecting the presence of an Stx in a biological sample, comprising:
contacting the biological sample with a hop bract tannin; and
detecting a macromolecular complex between the Stx and the hop bract tannin.

30. The method of claim 29, wherein detecting comprises detecting a precipitate comprising the complex.

31. The method of claim 29, wherein detecting the macromolecular complex between the hop bract tannin and the Stx comprises detecting an electrophoretic pattern associated with the presence of the macromolecular complex in the sample.

32. The method of claim 29, wherein the hop bract tannin serves as a bioreceptor of a biosensor and detecting comprises measuring a change in a property of a transducer of the biosensor.

33. The method of claim 29, wherein the hop bract tannin is a polycatechin between a 10-mer and a 30-mer.

34. The method of claim 29, wherein the polycatechin has the forumla 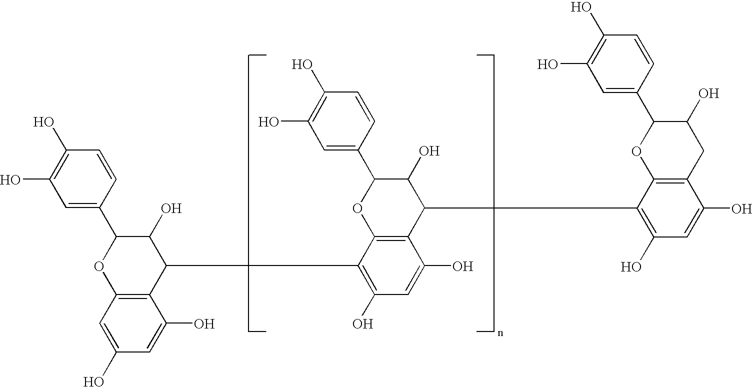
where n=8 to 28, or 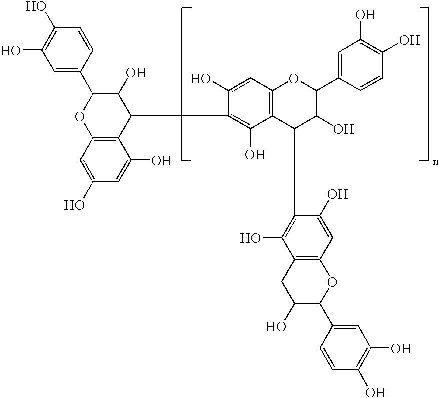
where n=8 to 28.

35. The method of claim 29, wherein the hop bract tannin comprises a fraction isolated from a hop bract extract.

36. The method of claim 35, wherein the fraction has a weight-average molecular mass between 5 kDa and 30 kDa.

37. A method for isolating and purifying Stx-binding polyphenols, comprising:
contacting a mixture comprising a Stx-binding polyphenolic compound isolated from Humulus lupulus with an Stx to form a macromolecular complex between the compound and the Stx;
isolating the macromolecular complex; and
separating the polyphenolic compound from the macromolecular complex to obtain a purified sample of the polyphenolic compound that binds Stx.

38. The method of claim 37, wherein the Stx is coupled to an activated chromatographic matrix.

39. The method of claim 37, wherein the Stx comprises he bioreceptor of a biosensor.

40. The method of claim 38, wherein the Stx is Stx1.

41. A method for prophylatic or post-exposure treatment of an inhaled Stx comprising administering a therapeutically effective amount of hop bract tannin intranasally to a subject.

42. A biosensor, comprising:
a hop bract tannin as a bioreceptor, and
a transducer.

43. The biosensor of claim 42, wherein the hop bract tannin is a polycatechin between a 10-mer and a 30-mer.

44. The method of claim 43, wherein the polycatechin has the forumla 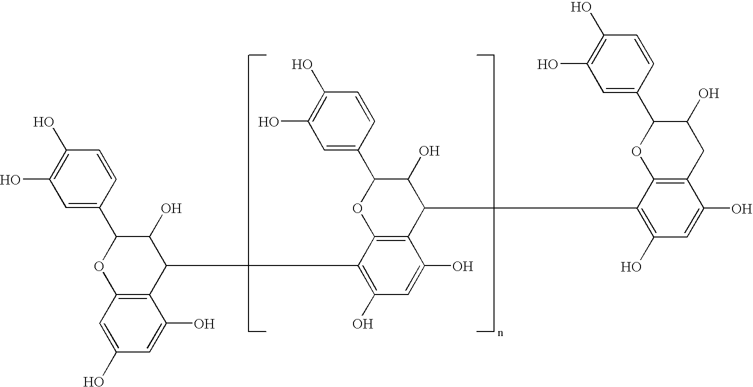
where n=8 to 28, or 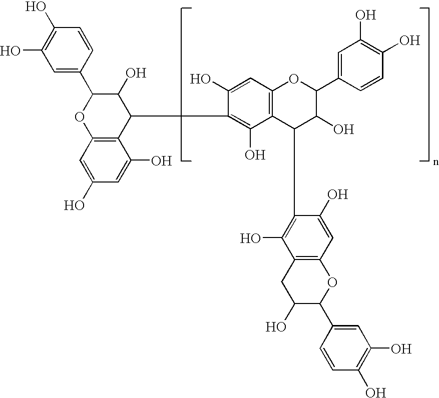
where n=8 to 28.

45. The method of claim 42, wherein the hop bract tannin comprises a fraction isolated from a hop bract extract.

46. The method of claim 45, wherein the fraction has a weight-average molecular mass between 5 kDa and 30 kDa.

47-57. (canceled)

58. A method for neutralizing a bacterial toxin, comprising:
providing a hop bract tannin; and
contacting the bacterial toxin with the hop bract tannin to neutralize the toxin.

59. The method of claim 58, wherein the bacterial toxin is selected from the group consisting of Shiga toxins and cholera toxins.

60. The method of claim 58, wherein the hop bract tannin comprises a subfraction having a weight-average molecular weight from 5 kDa to 30 kDa.

61. The method of claim 58, wherein the hop bract tannin comprises a polycatechin selected from the group of 10-mers to 30-mers, and mixtures thereof.

62. An isolated polyphenolic component of a high molecular weight fraction of a hop bract extract, the high molecular weight fraction having a weight average molecular weight of greater than 5 kDa.

63. A subfraction of a high molecular weight fraction of a hop bract extract, the high molecular weight fraction having a weight average molecular weight of greater than 5 kDa.

64. The subfraction of claim 63, wherein the subfraction has a weight average molecular weight range selected from the group consisting of 5 kDa-30 kDa, 5 kDa-10 kDa, 5 kDa-8 kDa, 8 kDa-30 kDa, 8 kDa-10 kDa and 10 kDa-30 kDa.
